(19) United States
(12) Patent Application Publication
(10) Pub. No.: US 2008/0221677 A1
(43) Pub. Date: Sep. 11, 2008

(54) Artificial eye socket system and method

(75) Inventor: Antonio R. Alfaro, Upper Black Eddy, PA (US)

Correspondence Address: JOHN W. GOLDSCHMIDT, JR. ESQUIRE;DILWORTH PAXON LLP, 3200 MELLON BANK CENTER, 1735 MARKET STREET, PHILADELPHIA, PA, 19103, US

(73) Assignee: Tohickon Corporation, Ottsville, PA (US)

(21) Appl. No.: 12/075,409

(22) Filed: Mar. 10, 2008

Related U.S. Application Data
(60) Provisional application No. 60/906,004, filed on Mar. 8, 2007.

(51) Int. Cl. A61F 2/16; A61F 2/28

(52) U.S. Cl. 623/6.64; 623/16.11

(57) ABSTRACT

An artificial eye socket for use in the mounting of artificial eye is disclosed. The artificial eye socket is of substantially unitary construction and made from a flexible material such as rubber. The artificial eye socket comprises an outer base surface; an inner base surface; a substantially contiguous outer sclera surface which extends from the outer base surface to an iris knife edge to and from an iris opening in said socket; and a substantially contiguous inner cavity surface which extends from said inner base surface to said iris knife edge to form said iris opening in said socket and a cavity within said socket. An artificial eye may be inserted into said cavity and secured in a substantially fixed position by the inner base surface and the cavity surface. Methods for manufacturing and mounting the artificial eye socket and cleaning the surface of the artificial eye are also disclosed.

### CROSS REFERENCE TO RELATED APPLICATION

This application claims priority to U.S. Provisional Patent Application Ser. No. 60/906,004, filed Mar. 8, 2007, the entirety of which is incorporated herein by reference.

### FIELD OF INVENTION

The present invention is generally directed to an artificial eye socket and method for mounting artificial eyes. More specifically, this invention is directed to an apparatus and method for mounting artificial eyes in taxidermy, doll making, sculptures and related applications, as well as the product resulting from the manufacturing process.

### BACKGROUND OF THE INVENTION

The construction of high quality artificial manikins having life-like characteristics (such as, for example, taxidermy mounts, dolls, stuffed animals, clothing mannequins, artistic sculptures, and the like) requires extraordinary skill, and can be extremely difficult, expensive, and time consuming. Each component, such as the eyes, skin, hide, hair, teeth and the like, must be accurately depicted in order for the manikin to be acceptable in the marketplace. Yet accurately depicting each such component often requires a high degree of technical expertise in both design and manufacturing.

One such component which presents unique difficulties in production and mounting is the manikin's eyes. Artificial eyes may be made from many materials; however, glass provides a superior substrate material because of its reflective properties and resistance to scratching. Nevertheless, artificial glass eyes are not without drawbacks in their production and in mounting them to a manikin. In this regard, artificial glass eye construction requires significant artistic skill, time, anatomical knowledge, and experience to produce a high quality artificial eye. Typical artificial glass eyes, such as taxidermic and doll eyes have a generally hemispherical or aspherical outer surface. The eyes are usually hollow having a convex outer surface and a flat or concave inner surface or back. Markings are typically placed on the concave back of the eye blank to simulate the components of a real animal, human or even fanciful eye. A realistic artificial eye includes the common visual eye elements for each particular application, such as for example, those visible features of an animal, fish, human, teddy bear, and the like. A typical artificial eye contains, for example, sclerotic capsule or base, pupil, iris, and cornea elements. Thus, the artificial eye design and manufacturing process requires that when assembled, the combined components create a realistic artificial eye, and that the elements are able to efficiently and correctly form together during manufacturing or production.

The design, manufacture and mounting of realistic artificial eyes are historically slow and tedious, and require highly trained artists and technicians. Artificial glass eye construction and mounting processes involve a number of interrelated factors, including: the ability to produce a variety of designs, the materials used, the manufacturing processes and the level of skill required to produce and mount anatomically realistic eyes. Accordingly, along with the artistic need to depict realistic eyes, these factors must all be considered in the design and mounting of artificial eyes. As described below, current artificial glass eye construction and mounting methods are technically complicated, time consuming and produce inferior results.

The problems encountered in producing and mounting artificial eyes are well illustrated, by way of non-limiting example, by considering the construction and mounting methods currently employed in fish taxidermy. In this regard, it should be noted that many artificial fish eyes sold today do not represent the entire eye; rather they represent only the iris of the eye.

Fish taxidermy is unique in many ways from taxidermy of mammals, birds, and reptiles. Generally, taxidermy utilizes the actual skin of a mammal, bird, or reptile to reproduce a lifelike representation of the animal. In contrast, some fish taxidermy is not actually taxidermy, but a reproduction of the fish using casting techniques and artificial materials. In this regard, fish taxidermy is broken into two main categories: (i) skin mounts; and (ii) reproductions.

In either case, it is no longer common practice to use the actual fish head. Even in skin mounting the skin is used, but the head is cast and a mold is made of the head so that an artificial casting of the head consisting of resin may be made. The reproduction of the head is then used in conjunction with the actual skin of the fish being laid over a polyurethane fish body. Polyurethane fish bodies are available through taxidermy supply houses. Alternatively, custom made fish bodies may be made out of Styrofoam by a taxidermist. Generally, the actual heads are no longer used in fish mounts because too many oils are present in the head and it is very difficult to remove the oils so that there will be no oily leakage after the fish mount has been completed.

The reproduced head casting is generally formed into a hollow shell resembling the actual fish's bead. The eye socket opening is formed by grinding away the eye socket opening in the head casting using a Dremel or similar rotary tool.

The taxidermist must then mount the eye in the socket by using clay and/or or epoxy. If glue or epoxy is used, the glue or epoxy may become brittle and the artificial eyes may fall out of finished mounts. In addition, clay may be used because it is pliable and easily shaped into the desired form. Shaping is important inasmuch as the taxidermist uses the clay not only to mount the eye, but also to form the sclerotic capsule of the eye.

In employing current eye mounting methods, shaping the socket properly and mounting the eye symmetrically with the opposite eye often proves to be a time consuming and delicate procedure. Experienced commercial taxidermists may take up to fifteen minutes or more to set or mount a single eye. In this regard, special attention must be paid to defining a properly shaped iris which is also the appropriate size. Also, where the clay sclera meets the fish head there must be a proper intersection. In this regard, the intersection cannot blend smoothly as the assembly is intended to represent an eye sitting in a socket. Fish have no eyelids, so the intersection between the sclera and the fish head is unique to fish. Once the eye has been mounted and intersection has been completed, the assembly must completely dry and set. However, as the assembly dries and sets, shrinking of the clay may occur causing shifting of the mounted eye as well as possible separation of the clay from the eye, particularly in the area of the sclera intersection.

After the eyes are set and all other taxidermy setting procedures are completed (such as, for example, mounting of artificial fins, detail work, habitat, etc.), the fish is painted. The skin generally retains some color but some of the color must be augmented by airbrushing. Also, the head must be painted to match the fish skin. In the case of a reproduction, the entire fish must be painted to match specifications established by photographs or otherwise by the client. In this regard, the sclera of a fish is not typically white like a human sclera. Rather, the fish sclera generally matches the skin color and pattern of the fish.

In painting the fish head and eye area, the entire eye, comprising clay and the glass eye, is covered in paint. The glass eye must then be cleaned without damaging the paint or clay of the clay sclera. The cleaning process is generally carried out by carefully cleaning the eye with solvents and by scraping the area with a knife or modeling tool. It is very easy at this point in known mounting processes to damage the clay socket with the solvents or by scraping, particularly where the socket meets the glass to form the visible portion of the iris.

Attempts have been made to overcome the shortcomings in mounting artificial fish eyes described above. In one case, a rubber socket system was developed for the Tohickon American Series 135 fish eyes. This system utilizes a pre-formed rubber socket into which the artificial eye may be inserted instead of using the traditional clay mounting and shaping techniques described above. First, the socket may be mounted in the cast head eye opening. The glass eye may then be glued to the pre-formed socket after painting is completed, supposedly eliminating the need to form an eye socket by hand and eliminating the cleaning process. However, this system has proven to be problematic because the iris portion of the socket could be easily distorted in the cast head eye opening during the mounting process. In this system, distortion of the socket typically occurs due to uneven pressure on the socket caused by being installed in an oddly shaped eye socket area in the cast head. In this regard, each fish differs in the size and shape of the eye opening in its head. Thus, this socket system is not readily adaptable to the wide variations in the size and shape of fish eye sockets which occur in nature.

This socket system may also be is used with the eye pre-installed with glue into the rubber socket. The fish may then be painted normally and the eye cleaned the same way it would be utilizing traditional clay mounted socket techniques. However, problems still remain with this system inasmuch as the taxidermist must still clean the eye right up to the edge of the socket without damaging the paint on the iris portion of the socket. In addition, and because of the nature of the uneven pressures on the socket, and the fact that the mating surface of the socket and eye is held together by glue, enough distortion is possible to separate a portion of the mating surface between the edge of the socket and the surface of the eye causing an unnatural void or gap between the eye and socket. Furthermore, inasmuch as the eyes are glued into the socket, as the glue dries the glue becomes brittle and the eyes tend to fall out of the sockets.

Additionally, the design of the American Series 135 fish eye used in combination with this rubber socket system has gross shortcomings in anatomical correctness. Specifically, the design of this socket system does not allow for the proper rotation of the eye set for a fish that is looking forward and/or down as is the common eye orientation for a fish in nature.

Another attempt to overcome the shortfalls encountered in the mounting of artificial fish eyes, is the Wayne Cooper Flex-Eye. This eye is made entirely of plastic and includes a socket area complete with coloration. In this device, the plastic of the eye/socket assembly is intended to be flexed by squeezing and then inserted into the head casting from the front of the casting. However, while touted as being flexible the plastic assembly is not nearly as pliable as necessary to mount the eye/socket in the fish casting. One must exert such a great deal of force to flex the assembly that many taxidermists merely trim the assembly to fit the socket opening in the casting, thereby defeating the many of the benefits of having a pre-formed socket and eye assembly.

Moreover, the coloring of the eye the Wayne Cooper Flex-Eye lacks the requisite anatomical correctness demanded by the industry. In this regard, the coloration shows a clear “dot” pattern which is not life-like at all. Also, the sclera color is pre-printed on the eye/socket assembly so that the taxidermist must paint the fish to match the pre-printed colors, rather than painting the fish to custom client specifications.

Finally, the plastic of the eye/socket assembly is easily scratched, and may rapidly degrade particularly if exposed to cleaning solvents which are used in taxidermy practice. Generally, and particularly in the eye regions, plastic does not have the brilliance, reflectivity, chemical resistance, or depth of glass. Thus, this device does not meet the demands of the industry to provide an anatomically correct and easily assembled artificial eye mounting system.

As the techniques utilized in producing anatomically correct artificial eyes of higher and higher quality have rapidly improved, it has been discovered that it is actually more difficult to set an anatomically correct artificial eye into a fish casting than it is to set other less correct eyes that are currently on the market.

Thus, there remains a need for an apparatus, system and method for mounting artificial eyes in various mounts, such as those used in taxidermy and other applications requiring anatomically realistic eye, without the need for the utilization of highly trained artisans and/or technicians. In addition, the need exists for a mounting system which provides consistent and predictable finished products, while reducing cost and production time.

### SUMMARY OF THE INVENTION

This invention is directed to an artificial eye socket and method for mounting artificial eyes. More specifically, this invention is directed to an apparatus, system and method and for mounting artificial eyes used in taxidermy, doll making and related applications. Such eyes may be adjusted during and, optionally, after the mounting process is completed to create a natural and realistic effect particularly when the artificial eye is made from a glass or glass-like substrate. In addition, when glass or glass-like artificial eyes are employed, the glass eye provides varying thicknesses of material which creates a more lifelike refraction of color.

As mentioned above, as the artificial eyes which are produced become more anatomically correct, it becomes more difficult to position and set the anatomically correct eye into a fish head casting than it is to set other less anatomically correct eyes that are currently on the market. Fundamentally, the instant invention addresses this problem by employing a highly flexible eye socket made of rubber or other appropriate material which exhibits the desired flexibility and other properties disclosed herein. One significant feature of the eye socket of this invention, beyond the improvements in design shape, is that no glue, epoxy or other adhesive is required to hold the artificial eye in place in the eye socket. This feature eliminates the problem of the eyes falling out of finished mounts due to the glue drying out and becoming brittle over time. The eye socket of this invention also provides an excellent surface to permit the blending of the painted surfaces from the surface of the fish head casting to the eye socket surface to the surface of the artificial eye itself. The eye socket creates a particularly superior transitional surface between the eye socket and the artificial eye itself to permit the natural and life-like blending of surface colors and details due to the knife edge formed by the sclera portion of the eye socket where the artificial eye and socket meet. In this regard, previous attempts at creating an eye socket for the mounting of artificial eyes did not have such a knife edge and, thus, the colors and details of the surface paint did not blend as well. Furthermore, an unnatural gap between the artificial eye and the socket is often present with other eye socket devices thus producing an inferior final product. Moreover, because the artificial eye is not glued or otherwise affixed to the socket of this invention, the artificial eye may be adjusted or otherwise positioned as necessary after the eye is placed in the socket.

In addition, no eye socket or socket system has been developed where the socket envelopes the eye so as not to require gluing. The eye socket of this invention envelopes the artificial eye in this manner. Also, because the socket envelopes the eye it completely covers the back of the eye, allowing for the use of inks (such as pad printing inks) that are more prone to delamination when exposed to epoxies and other solvent materials in the painting of the pupil, iris, cornea and other details on the reverse or inner surface of the artificial eye.

### BRIEF DESCRIPTION OF THE DRAWINGS

Understanding of the present invention will be facilitated by consideration of the following detailed description of the embodiments of the present invention taken in conjunction with the accompanying drawings, in which like numerals refer to like parts, and wherein:

FIG. 1 is a side elevation view of a fish head casting into which the eye socket system of this invention has been mounted, and wherein the broken line depicts the position of the outer edge of the artificial eye socket of this invention beneath the surface of the casting.

FIG. 2 is a plan view of the bottom surface of the artificial eye socket, depicting the number 16 as size of the socket and an arrow or carrot pointing in the direction of the front of the casting.

FIG. 3 is a plan view of the top surface of an artificial eye inserted into the eye socket of this invention.

FIG. 4 is a plan view of the top surface of the eye and socket of FIG. 3, and indicating that the artificial eye may be rotated when mounted in the eye socket.

FIG. 5 is a side elevation view of the eye socket of this invention.

FIG. 6 is a front elevation view of the socket of FIG. 5.

FIG. 7 is a cross sectional side elevation view of the socket of FIG. 5 before the artificial eye has been inserted.

FIG. 8 is a cross sectional side elevation view of the socket of FIG. 7 after the artificial eye has been inserted.

FIG. 9 is a cross sectional front elevation view of the socket of FIG. 6 before the artificial eye has been inserted.

FIG. 10 is a cross sectional front elevation of the socket of FIG. 9 after the artificial eye has been inserted.

FIG. 11 is a cross sectional side elevation view of a socket of this invention depicting a male alignment notch in the socket, and a side elevation cross section of an artificial eye also depicting the female alignment notch prior to insertion of the artificial eye into the socket.

FIG. 12 is a cross sectional side elevation view of the socket and artificial eye of FIG. 11 after insertion of the artificial eye into the socket.

FIG. 13 is a cutaway view of the front or leading side view of a socket of this invention depicting a male alignment notch of the socket, and a front or leading side view of an artificial eye, also depicting a female alignment notch prior to insertion of the artificial eye into the socket.

FIG. 14 is a cutaway view of the socket and artificial eye of FIG. 13 after the insertion of the artificial eye into the socket.

Additional documents attached hereto constitute additional teachings included in the present invention, including Appendix A and Appendix B hereto each of which comprise photographs and other descriptive material which depicts additional features and embodiments of the instant invention, all of which is incorporated herein by reference.

### DETAILED DESCRIPTION

It is to be understood that the figures and descriptions of the present invention have been simplified to illustrate elements that are relevant for a clear understanding of the present invention, while eliminating, for the purposes of clarity, many other elements which may be found in the present invention. Those of ordinary skill in the pertinent art will recognize that other elements are desirable and/or required in order to implement the present invention. However, because such elements are well known in the art, and because such elements do not facilitate a better understanding of the present invention, a discussion of such elements is not provided herein.

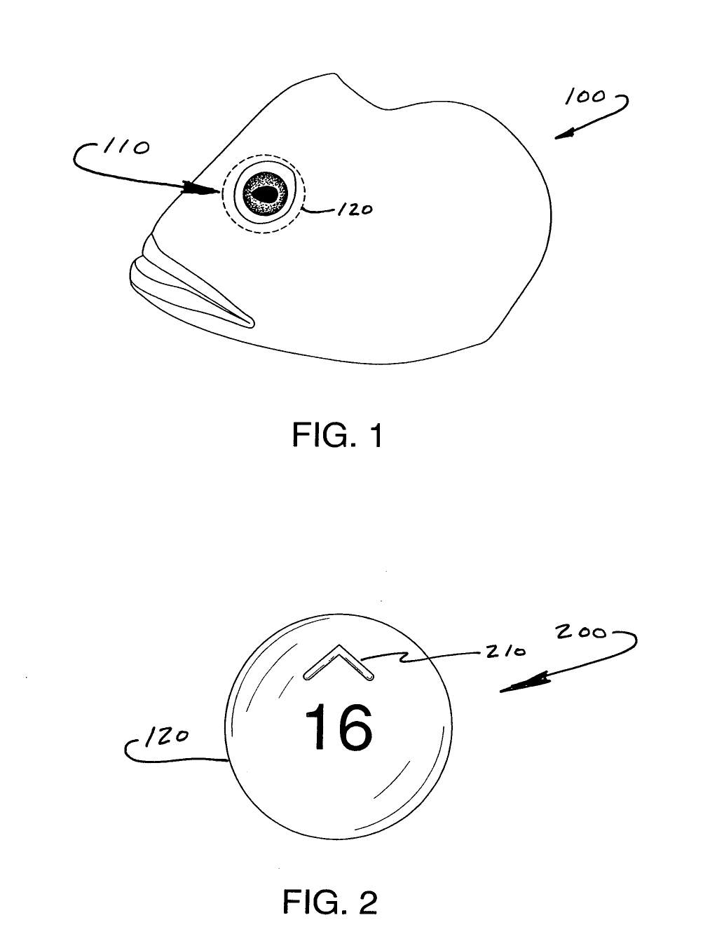

Turning now to FIG. 1 one embodiment of the eye and socket system 110 of the instant invention is depicted as being mounted to a fish head casting 100. The position of the outer edge of the bottom base of the artificial eye socket of this invention located beneath the surface of the casting is depicted in phantom as numeral 120.

FIG. 2 depicts an embodiment of the bottom surface of the artificial eye socket 200. The number 16 indicates the size of the socket, and arrow or carrot 210 indicates the direction in which socket 200 should face when positioned in casting 100. For example, in the embodiment shown in FIG. 2, arrow 210 should point in the direction the front of fish casting 100 when socket 200 is mounted in casting 100.

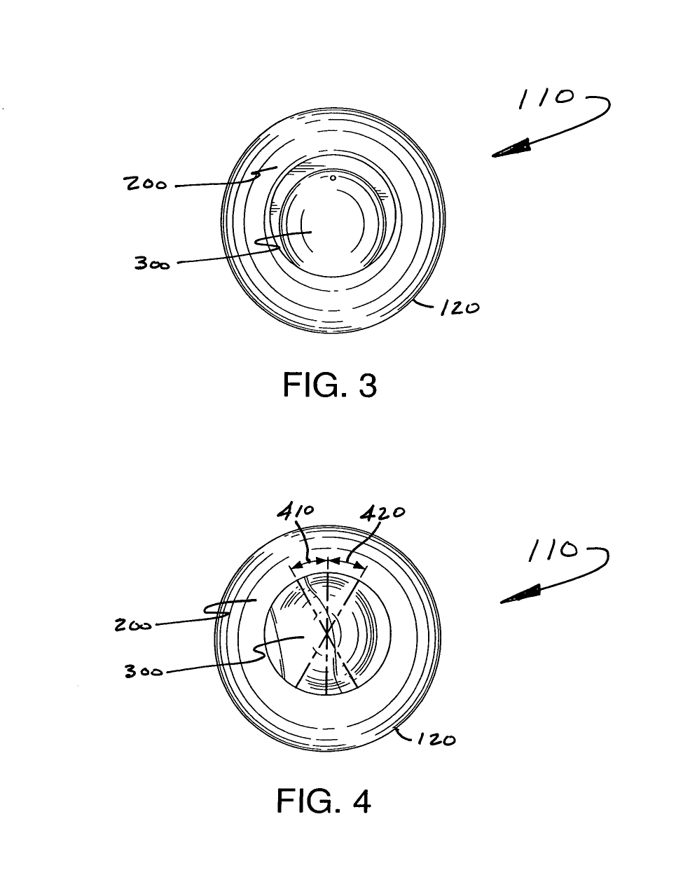

FIG. 3 depicts a plan view of an embodiment of eye/socket assembly 110. Shown is the top surface of artificial eye 300 inserted into eye socket 200.

FIG. 4 depicts a plan view of the top surface of eye and socket assembly 110 of FIG. 3. Arrows 410 and 420 indicate that artificial eye 300 may be rotated and otherwise adjusted after eye 300 has been mounted in eye socket 200.

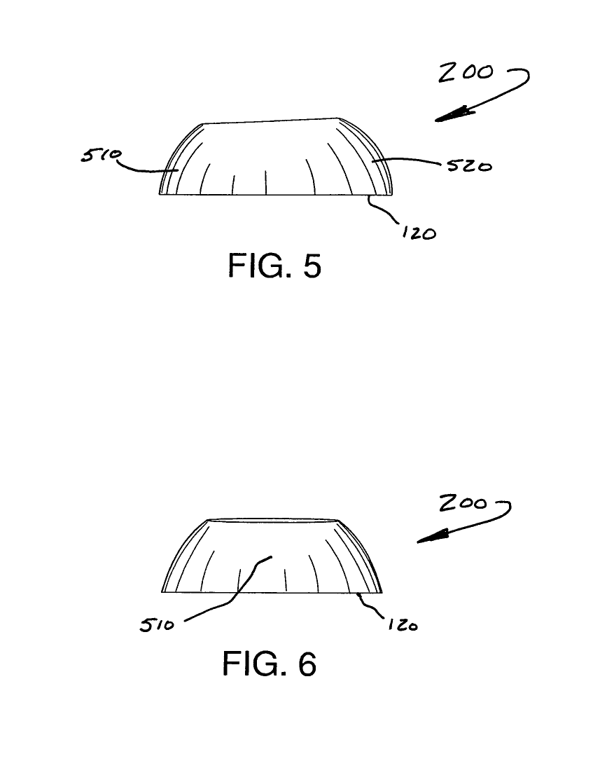

FIG. 5 depicts a side elevation view of an embodiment of eye socket 200 of this invention. The left side of socket 200 is front or leading side 510 of socket 200. When properly oriented in casting 100, leading side 510 would substantially face the front or leading (mouth) end of casting 100. In the embodiment shown in FIG. 2, arrow 210 would also point in the direction of the leading end of casting 100. Furthermore, in the embodiment shown in FIG. 5, the right side of socket 200 is the rear or trailing side 520 of socket 200.

FIG. 6 depicts an elevation view of socket 200 as viewed from the front or leading side 510 of socket 200 shown in FIG. 5.

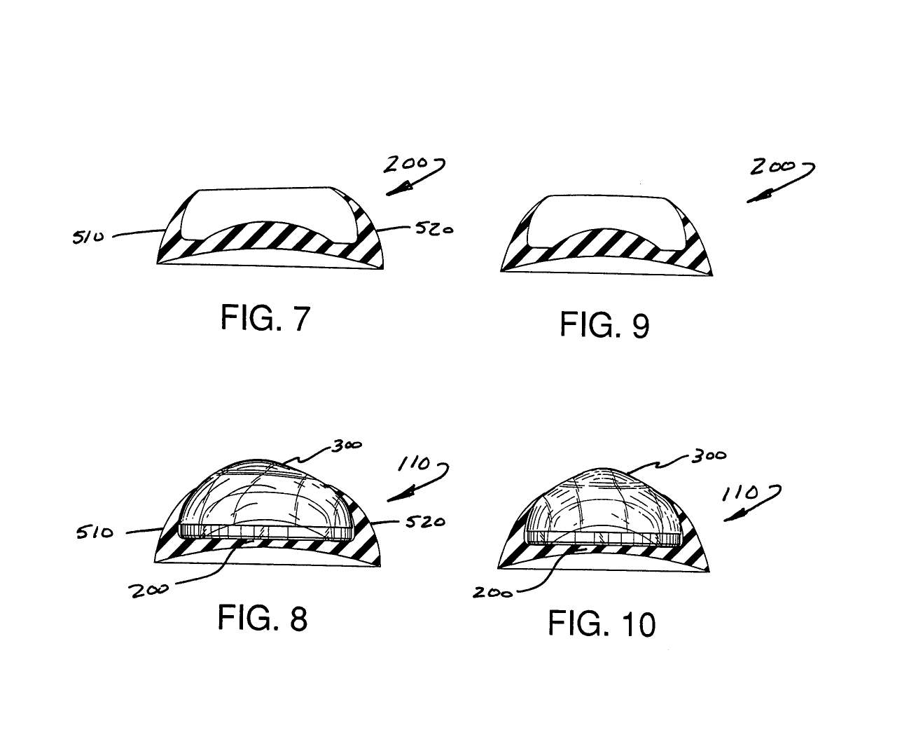

FIG. 7 depicts a cross sectional side elevation view of socket 200 of FIG. 5 before artificial eye 300 has been inserted.

FIG. 8 depicts a cross sectional view of socket 200 of FIG. 7 after artificial eye 300 has been inserted.

FIG. 9 depicts a cross sectional elevation view of front side 510 of socket 200 of FIG. 6 before artificial eye 300 has been inserted.

FIG. 10 depicts a cross sectional view of socket 200 of FIG. 9 after artificial eye 300 has been inserted.

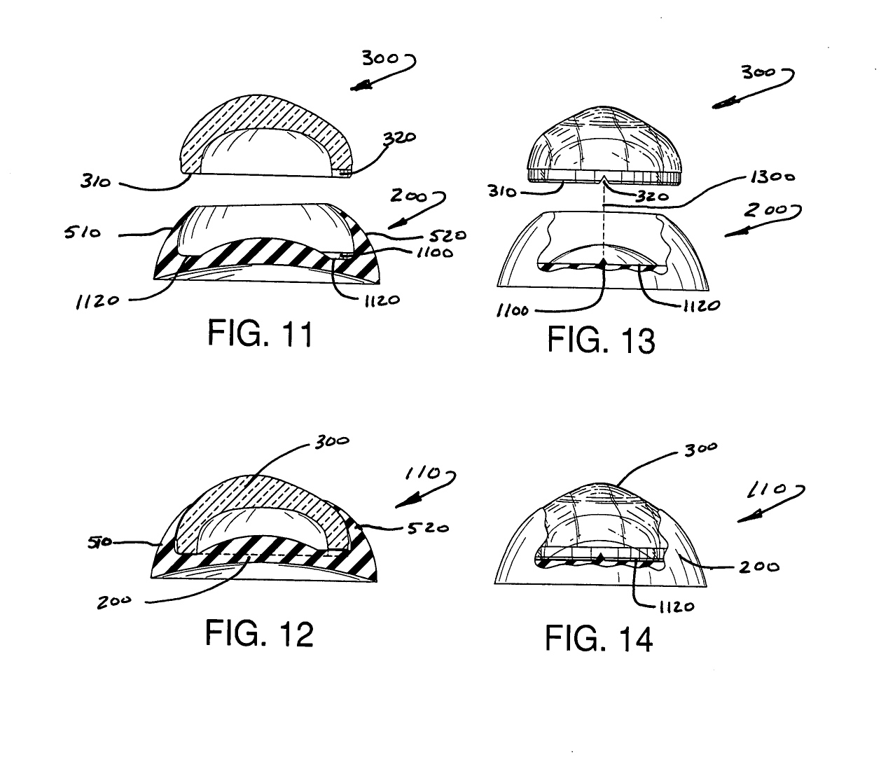

FIG. 11 is a cross sectional side elevation view of an embodiment of socket 200 prior to insertion of artificial eye 300 into socket 200. Also depicted in FIG. 11 is male alignment notch 1100 located on inside base 1120 of socket 200 where base 310 of eye 300 contacts inside base 1120 of socket 200. Also shown in FIG. 11 is the side elevation cross section of artificial eye 200 depicting female alignment notch 320 located in base 310 of artificial eye 300 prior to the insertion of artificial eye 300 into socket 200. Although male and female alignment notches 110 and 320 may be positioned in any location so as to substantially align eye 300 in a desired orientation, in this embodiment, male alignment notch 1100 and female alignment notch 320 are generally oriented toward rear or trailing side 520 of socket 200. In yet another embodiment of the instant invention, and as further described below, FIGS. 12 and 13 depict male and female alignment notches 1100 and 320 which are generally oriented toward the front or leading side 510 of socket 200.

FIG. 12 depicts a cross sectional of socket 200 of FIG. 11 after insertion of artificial eye 300 into socket 200.

The embodiment shown in FIG. 13 is a cutaway view of front or leading side view 510 of another embodiment of socket 200. FIG. 13 further depicts is male alignment notch 1100 of socket 200, wherein notch 1100 is substantially located on inside base 1120 of socket 200 where base 310 of eye 300 substantially contacts inside base 1120 of socket 200. Also shown in FIG. 13 is the front or leading side view of the artificial eye 300 depicting female alignment notch 320 located in base 310 of artificial eye 300 prior to the insertion of artificial eye 300 into socket 200. Broken line 1300 depicts the path upon which male and female notches 1100 and 320 are substantially aligned.

FIG. 14 depicts a cutaway view of front or leading side 510 of socket 200 of FIG. 13 after eye 300 is inserted in socket 200. Also depicted in FIG. 14 is male alignment notch 1100 of socket 200 located on inside base 1120 of socket 200 where base 310 of eye 300 contacts inside base 1120 of socket 200. Also shown in FIG. 14 is the front or leading side of artificial eye 300, comprising female alignment notch 320 located in base 310 of artificial eye 300, after the insertion of artificial eye 300 into socket 200. In this view, male and female alignment notches 1100 and 320 are engaged.

As depicted in FIG. 13, artificial eyes 300 utilized in one embodiment of the instant invention have a female alignment notch 320 incorporated into the edge of base 310 of eye 300 which indicates to the user the position of the front of eye 300, although notch 320 may be located in any desired position. As also shown in FIG. 13, eye socket 200 of this embodiment of the invention comprises male alignment notch 1100 formed in the surface of base or bottom surface 1120 of the inside of socket 200. Male alignment notch 1100 in socket 200 also acts as a visual indicator for the user of where to position eye 300 in socket 200. As shown in FIG. 14, male and female alignment notches 1100 and 320, respectively, engage when eye 300 is properly seated and positioned in socket 200. However, it should be noted that the position of eye 300 maybe adjusted in socket 200, as desired, after eye 300 has been inserted in socket 200. Moreover, it should be generally noted that male and female alignment notches 1100 and 320 may be positioned in any location on socket 200 and eye 300 respectively so as to promote (i) the substantial engagement of notches 1100 and 320 when eye 300 is seated in socket 200, (ii) the desired orientation of eye 300 in socket 200; and/or (iii) the alignment of socket 200, eye 300 and or eye/socket assembly 110 in casting 100.

The sockets of the embodiment shown in the figures comprise an opening also referred to herein as the iris opening through which the eye is inserted. The opening is substantially oval in shape and is considerably smaller than the outside shape of the artificial eye. The oval opening also roughly defines the iris of the eye. The size and shape of the opening of the socket provides a tight fit around the eye so that the eye does not fall out and eliminates the need for glue or other adhesives to secure the eye in place. Moreover, a proper oval iris may be defined by the oval opening, even over an uneven shape such as the forward leaning artificial eye design of the 137 C-BRYTE eye, also depicted in U.S. Patent No. D 529,241 S, the entirety of which is incorporated herein by reference.

It should also be noted that in the instant invention, even when uneven pressures are present on the outside of the socket which may slightly distort the iris shape formed by the opening in the socket, no void is created between the rubber knife edge of the socket and the adjoining surface of the artificial eye as the rubber like material is flexible and pliable enough to simply slide along the surface of the eye (the artificial eye preferably being made of a glass or glass like material), therein slightly opening the iris opening in the socket.

The material from which the socket is constructed is preferably soft, flexible and pliable enough to permit the snug fit between the socket opening and the surface of the eye described above. Soft rubber or rubber like materials may be utilized. This softer material allows a taxidermist or other user who wants to mount the eye from the front of a fish head casting to secure the eye into place. The eye socket may be trimmed or otherwise shaped to accommodate the eye or to mount the socket to the fish head casting.

The eye may also be mounted from behind the fish head casting by placing the eye and socket up inside the head casting and positioning the eye/socket assembly in the opening using clay, epoxy, or glue.

In addition, the softer more flexible eye socket material is also advantageous when it comes to cleaning a mounted glass eye. As mentioned above, the transitional area where the sclera meets the iris is critical. When cleaning, no paint may be left on the surface of the glass eye, and no paint may be removed from the socket, whether the surface material is clay or rubber. When the sclera is made of clay or hard plastic or rubber, extreme care must be exercised by the taxidermist not to disturb the clay or any paint on the hard plastic or rubber while cleaning the glass up to the clay or hard rubber edge.

By contrast, the eye socket of the instant invention permits artificial glass eyes to be completely cleaned without harming the flexible rubber of the eye socket or the paint applied to the socket. In this regard, the taxidermist may use a modeling tool to scrape the paint away at the meeting point of the glass eye and the knife edge formed at the opening of the rubber eye socket by sliding the modeling tool underneath the lip or knife edge of the socket. This is possible due to the increased flexibility of the socket material as well as the functionality of the shape of the socket which holds the eye in place and by using no glue or adhesive to hold the eye in place. Thus, with the eye socket of the instant invention, a completely clean and perfectly shaped iris can be produced without damaging the sclera surface of the eye socket or the artwork applied to it. In addition, the socket of the instant invention permits the taxidermist to choose the sclera coloration such that the depth, color, clarity, durability and anatomical correctness of a glass eye may be utilized to produce a superior finished product.

Thus, the eye socket system of the instant invention may comprise one or more of the following features:

1. the eye socket of this invention may hold the artificial eye in place without the aid of glue or other adhesive such that the eye cannot fall out of the socket;

2. no unnatural voids may be present in the transitional area between the knife edge of the socket and the surface of the eye;

3. cleaning of the surface of the artificial eye may be simplified. The use of a modeling tool under the lip or knife edge where the rubber socket meets the surface of the eye may allow a taxidermist to clean the iris of the eye and beyond without substantially damaging the socket or the paint on the socket;

4. the artificial eye may be positioned after the eye is placed in the eye socket because no glue or other permanent adhesive is used to affix the eye to the socket; and/or

5. the eye/socket assembly may produce an anatomically correct artificial eye in shape, color and other details.

Embodiments of the apparatus, system and method of the instant invention are also disclosed in Appendix A and Appendix B, attached hereto. One embodiment of the method of making the artificial eye sockets of this invention is depicted in Appendix A. See in particular A-1 through A-14. More specifically, this embodiment depicts a method for making an artificial eye socket for a fish head casting. Nevertheless, the methods disclosed may also be adapted to numerous other applications requiring the mounting of artificial eyes such as for example animals, mammals, dolls, plush toys, manikins, taxidermy mounts and the like.

Referring again to Appendix A, there is disclosed a method for constructing a master socket mold which may be used to produce the artificial eye sockets of this invention. See A-1 through A-12.

Also disclosed in Appendix A is a method for producing the artificial eye sockets of this invention from the master socket mold shown in A-1 through A-12. See A-13 and A-14.

A method for inserting an artificial glass eye into the rubber artificial eye sockets (shown in A-13 and A-14) is also disclosed in Appendix A. See A-15 through A-19.

A method for mounting the artificial glass eye and socket assembly (shown in A-15 through A-19) from the inside of a cast fish head is also disclosed in Appendix A. See A-20 through A-26.

A method for painting or otherwise decorating a fish head casting having an artificial eye and socket assembly mounted therein (see A-20 through A-26) is also disclosed in Appendix A. See A-27 through A-37.

A method for cleaning an artificial glass eye in the artificial socket of this invention after a painting process (see A-27 through A-37) is also disclosed in Appendix A. See A-38 through A-43.

A method for completing the decoration of an artificial eye and socket assembly and fish head casting after a glass eye cleaning process (see A-38 through A-43) is also disclosed in Appendix A. See A-44 through A-47.

Another embodiment of a method for mounting the artificial eye and socket assembly of this invention is disclosed in Appendix B. More specifically, Appendix B discloses a method for mounting the artificial eye and socket assembly of this invention from the outside of a cast fish head. See generally B-1 through B-10.

A method for preparing a casting and modifying the artificial eye socket of this invention for mounting from the outside of the casting is disclosed in Appendix B. See B-1 through B-3.

A method for inserting and mounting the eye socket from the outside of the casting is also disclosed in Appendix B. See B-4 through B-9.

A method for inserting an artificial eye into the mounted artificial eye socket of this invention (see B-4 through B-9) is also disclosed in Appendix B. See B-10.

The artificial eye socket of the instant invention also allows for the use of a fewer number of artificial eye sizes while still providing for a full range of eye sizes in the finished product.

For example, artificial eye sizes used in fish taxidermy generally are available in 1 millimeter increments from about 8 millimeters up to and including about 18 millimeters. Traditional artificial eye mounting methods would require the taxidermist to use, for example, a 15 millimeter eye and mold the socket area with clay to accommodate the 15 millimeter eye with a proportionate iris opening. As discussed above, this process requires exceptional skill and is highly time consuming.

In contrast, the artificial eye socket of this invention may use a 16 millimeter artificial eye in a 16 millimeter socket wherein the 16 millimeter socket has been modified to have an iris opening in the socket sized to be proportionate to a 15 millimeter eye. Because the difference in curvature of the surface of a 15 millimeter and 16 millimeter eye is not visually perceptible to the unaided human eye after the eye/socket assembly of this invention is mounted to a casting, it is possible to use, for example, only even sizes of artificial eyes (e.g. 8 millimeter, 10 millimeter, 12 millimeter etc.)

The iris opening of the artificial eye socket may be varied by molding sockets with varying sizes of iris openings or, alternatively, trimming the iris opening to the desired size, so long as the socket maintains the capability of retaining the artificial eye in place.

For example, in this invention, a 16 millimeter socket may be sized with a 15 millimeter proportionate iris opening. A 16 millimeter artificial eye is inserted into the 16 millimeter socket. Because of the proportionate sizing of the iris opening, one perceives the 16 millimeter eye as being a 15 millimeter eye.

Alternatively, in the example above, the iris opening of a 16 millimeter socket having a 15 millimeter proportionate iris opening, may be trimmed back to a 16 millimeter iris opening. Thus, the same 16 millimeter molded socket and 16 millimeter artificial eye may be used to produce both 15 millimeter and 16 millimeter finished products.

It should be noted that the variations on the number and sizes of eyes and sockets used is principally limited by the ability of the socket to retain in position the artificial eye, and the human eye's ability to perceive the difference in curvature of the surface of various sizes of artificial eyes. Thus, it may be possible to use a single size of artificial eye in applications spanning two or more size ranges. For example, by adjusting the size of the iris opening, or other features of the artificial eye socket, it may be possible to use a 14 millimeter artificial eye in applications requiring 12 millimeter, 13 millimeter, 14 millimeter, 15 millimeter, and 16 millimeter artificial eyes.

The apparatus, system and methods described in detail herein are based upon the mounting of one type of eye to a fish head casting. As mentioned above, the principles described herein may be applied to many other applications in the field of taxidermy as well as in connection with products produced in unrelated fields such as dolls, plush toys, clothing mannequins, artistic sculptures, and the like.

While the foregoing invention has been described with regard to certain preferred embodiments, and certain details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Thus, such modifications and embodiments are also intended to fall within the scope of the appended claims.

What is claimed is:

1. An artificial eye socket comprising:
an outer base surface;
an inner base surface;
a substantially contiguous outer sclera surface which extends from said outer base surface to an iris knife edge to form an iris opening in said socket;
a substantially contiguous inner cavity surface which extends from said inner base surface to said iris knife edge to form said iris opening in said socket and a cavity within said socket;
wherein an artificial eye may be inserted into said cavity and secured in a substantially fixed position by said inner base surface and said cavity surface.

2. The artificial eye socket of claim 1, further comprising an artificial eye comprising an outer artificial eye surface and an artificial eye base surface having a female alignment notch therein, and wherein said artificial eye is positioned in said cavity.

3. The artificial eye socket of claim 2, wherein said artificial eye is substantially composed of glass or a glass-like material.

4. The artificial eye socket of claim 1, wherein said iris opening is substantially oval in shape.

5. The artificial eye socket of claim 1, wherein glue or other adhesive is not required to secure said artificial eye in said cavity.

6. The artificial eye socket of claim 2, wherein said inner cavity surface which is in substantial proximity to said iris knife edge is in substantial contact with said outer artificial eye surface.

7. The artificial eye socket of claim 6, wherein there is no separation between said inner cavity surface visible to the unaided human eye.

8. The artificial eye socket of claim 2, wherein said artificial eye may be positioned or repositioned in said socket.

9. The artificial eye socket of claim 1, where said inner base surface comprises a male alignment notch.

10. The artificial eye socket of claim 2, where said inner base surface comprises a male alignment notch which is substantially engaged with said female alignment notch of said artificial eye.

11. The artificial eye socket of claim 2, wherein said inner base surface comprises a male alignment notch which is not substantially engaged with said female alignment notch of said artificial eye.

12. The artificial eye socket of claim 2, wherein said socket and said artificial eye are decorated to produce a substantially anatomically correct finished product.

13. The artificial eye socket of claim 1, wherein said artificial eye socket is mounted to an animal, mammal, fish, doll, plush toy, or manikin.

14. The artificial eye socket of claim 1, wherein said artificial eye socket is mounted to an animal casting, mammal casting, fish casting, doll casting, sculpture casting, manikin casting or taxidermy mount.

15. The artificial eye socket of claim 2, wherein said artificial eye socket is mounted to an animal, mammal, fish, doll, plush toy, sculpture or manikin.

16. The artificial eye socket of claim 2, wherein said artificial eye socket is mounted to an animal casting, mammal casting, fish casting, doll casting, sculpture casting, manikin casting or taxidermy mount.

17. The artificial eye socket of claim 1, wherein said socket is substantially constructed from a rubber.

18. The artificial eye socket of claim 2, wherein said socket is substantially constructed from a rubber.

19. A method of making an artificial eye socket comprising the steps of:
depositing liquid plastic in a mold in the form of an artificial eye socket allowing the liquid plastic to cure; and
removing the formed artificial eye socket from said mold.

20. A method of mounting an artificial eye, said artificial eye having an outer surface, in a casting said casting having an inside, an outside and an eye socket opening, comprising the steps of:
inserting an artificial eye into a artificial eye socket to form an eye/socket assembly;
from said inside of said casting, positioning said eye socket assembly in said eye socket opening; and
affixing said eye/socket assembly in said position.

21. The method of claim 20, further comprising the step of:
painting or otherwise decorating said casting and eye/socket assembly.

22. The method for claim 21, further comprising the step of;
cleaning said surface of said eye of all paint and other decorative materials without disturbing the decorative features of said socket.

23. The method of claim 22, wherein said cleaning step further comprises the step of cleaning said surface of said artificial eye under said socket.

24. A method of mounting and artificial eye, said artificial eye having an outer surface, in a casting, said casting having inside, an outside and an eye socket opening, comprising the steps of;
from said outside of said casting, inserting an artificial eye socket in opening;
positioning said socket in said eye socket opening;
affixing said eye/socket assembly in said position; and
inserting said artificial eye in said socket.
